(19) United States
(12) Patent Application Publication
(10) Pub. No.: US 2002/0081365 A1
(43) Pub. Date: Jun. 27, 2002

(54) Method for the production of germ-enriched soybean material

(75) Inventors: Hirotoshi Ishida, Yokohama-shi (JP); Mamoru Ueno, Yokohama-shi (JP); Yuji Nakata, Yokohama-shi (JP); Akihiro Nakatani, Yokohama-shi (JP); Masaru Honda, Yokohama-shi (JP); Yoshio Shidou, Yokohama-shi (JP)

Correspondence Address: OBLON SPIVAK MCCLELLAND MAIER & NEUSTADT PC, FOURTH FLOOR, 1755 JEFFERSON DAVIS HIGHWAY, ARLINGTON, VA, 22202, US

(73) Assignee: Ajinomoto Co., Inc., Chuo-ku (JP)

(21) Appl. No.: 09/968,979

(22) Filed: Oct. 3, 2001

(30) Foreign Application Priority Data
Oct. 6, 2000 (JP) 2000-307046

(51) Int. Cl. A23D 9/00; A23L 1/20

(52) U.S. Cl. 426/601; 426/634

(57) ABSTRACT

The purposes of the present invention is to provide a method for the production of soybean material having a high germ concentration with a high yield, in which the germ is completely removed from the soybean (cotyledon), crushed cotyledon with a small diameter, which will be inevitably produced in the crudely crushing step of the soybean and can not be removed by the sieve having a square opening, is separated from the germ fraction, and mingling of the crushed cotyledon is prevented in order to maximize the yield of the germ that has scarcity value.

The present invention relates to a method for producing a soybean material having a germ concentration of 35-80 wt %, comprising crudely crushing a starting soybean material or its selected seeds from which foreign substances have been removed into a size of ½ or less of the original one, and separating and concentrating a soybean germ fraction by means of a sieve having an opening with 0.5-1.4 mm in width and with a ratio of length/width of 2 or more, oil and fat obtained from the soybean material produced according to the present invention or an isoflavone-rich product, food containing said oil and fat, and an agent for lowering cholesterol comprising as an effective component said oil and fat.

### TECHNICAL FIELD

The present invention relates to a method for producing germ-enriched soybean material, which is characterized in that a germ fraction (although the “germ” should be academically called a “hypocotyle”, the term “germ ” is used in the present specification in the same meaning as the hypocotyle) is sieved shape-dependently from a crudely crushed soybean.

### BACKGROUND ART

Like other oil seeds, soybean consists of each part of a cotyledon (ca.90%), a germ (ca.2%) and a hull (ca.8%). The soybean is used as material for soybean oil, after its hull is removed but the cotyledon and the germ that are rich in oil are not separated with each other.

When the oil is produced from the soybean, foreign substances such as stem, sheath and other seeds are first removed from a starting soybean material in a selection step in order to improve quality of a final oil product and defatted cake. Then, the material is provided with elasticity by being subjected to, for example, a heating treatment, crushed by means of a crushing roller or rubber roller and separated into the hull, cotyledon and germ parts. The hull, which is poor in oil and contains components such as a pigment that will adversely affect the quality of oil, is removed by means of a vibrating sieve or a sorting apparatus with air. The cotyledon and germ are flaked altogether to destruct their structures and to ease extraction of oil, followed by the extraction with n-hexane to give a crude oil, which will be finally purified to yield the soybean oil.

Japanese Patent Laid-Open Application Sho.59 (1984)-82063 and Japanese Patent Laid-Open Application Hei.11 (1999)-196803 disclose a method for obtaining a germ part from half-cut soybeans by means of a sieve or sorting with air. Japanese Patent Laid-Open Application Sho.56 (1981)-115836 discloses a method for the concentration of soybean germ by sorting crudely crushed soybeans with air and separating a fraction of 14-60 mesh by means of a sieve.

However, in these methods the crushing is controlled so that the soybeans will be crudely crushed only to a degree of their half-cut while preventing the production of crushed cotyledon with a small diameter as much as possible. And even if said crushed cotyledon is produced, mingling of the thus produced small-size cotyledon with a concentrated germ fraction will be prevented by the sieve. As a result, these methods have disadvantages that the germ attached to the half-cut soybeans will be lost.

On the other hand, if the soybeans are crushed more sufficiently to such a degree as smaller than their half-cut size in order to obtain a high yield in a conventional method wherein a square sieve is used, the crushed cotyledon with a small diameter will be mingled into the germ fraction, reducing the germ concentration. Thus, yield or concentration of the germ has to be sacrificed alternatively in the conventional methods.

The purposes of the present invention are therefore to provide a method for the production of soybean material having a high germ concentration with a high yield, in which the germ is completely removed from the soybean (cotyledon), and the mingling of the crushed cotyledon with a small diameter, which will be inevitably produced in the crudely crushing step of the soybean, is prevented in order to maximize the yield of the germ that has scarcity value.

The present inventors have studied to overcome the above problems, and have finally found that even the staring soybean material is crudely crushed to such a degree that the germ is completely removed from the cotyledon in a process of the separation of germ, size of the germ in its thickness direction is sufficiently smaller than that of the crushed cotyledon. Thus, it is found that shape of the mesh of a sieve is important by the present inventors.

### DISCLOSURE OF THE INVENTION

The present invention relates to a method for producing a soybean material having a germ concentration of 35-80 wt %, comprising crudely crushing a starting soybean material or its selected seeds from which foreign substances have been removed into a size of ½ or less, preferably of from ⅛ or more to ⅖ or less of the original one, and separating and concentrating a soybean germ fraction by means of a sieve having an opening with 0.5-1.4 mm, preferably 0.7-1.2 mm in width and with a ratio of length/width of 2 or more, preferably of 3-5.

Furthermore, the present invention relates to oil and fat obtained from the soybean material produced according to the present invention or an isoflavone-rich product, food containing said oil and fat, an agent for lowering cholesterol comprising as an effective component said oil and fat.

### BEST MODE FOR CARRYING OUT THE INVENTION

First, the starting soybean material (whole soybean) may be optionally selected by removing foreign substances (contaminants) such as stem, sheath, weeds, sands, metal particles and small stones.

Next, the thus selected starting soybean material (the selected seeds) is heated, dried, peeled or crudely crushed into cotyledon, germ and hull parts. The peeling or crushing may be carried out by means of friction, impact stress, shearing stress and the like, and any means known to those skilled in the art such as a crushing roller apparatus may be used for them.

The starting soybean material is crudely crushed into the size of ½ or less, preferably of from 1/16 or more to less than ½, more preferably of from ⅛ or more to ⅖ or less of the original one. The crude crushing into the size of ½ would cause loss of the germ due to its attachment to the half-cut soybean. The crude crushing into the size of less than ½ (i.e., containing substantially no half-cut soybeans) will make it easier to remove the germ from the soybean cotyledon. On the other hand, the crude crushing into the size of less than 1/16 would allow increase of an amount of the crushed cotyledon with a small diameter that are mingled into the concentrated germ fraction, reducing the germ concentration. Taking a balance between the yield and concentration of the germ into consideration, it is preferred to crudely crush the starting soybean material into the size of from ⅛ or more to ⅖ or less of the original one. These fraction values are determined based on measurement of their weight.

The desired product according to the present invention may be obtained by sieving the crudely crushed soybean (cotyledon, germ and hull) by using the sieve having an opening with 0.5-1.4 mm, preferably 0.7-1.2 mm in width and with a ratio of length/width of 2 or more, preferably 3-5 so that almost all of the crushed cotyledon will be left separated on the sieve.

The lower limit in width of the opening is attributed to the size of the germ in its thickness direction of about 0.5 mm. The upper limit of 1.4 mm in width of the opening has been determined since a greater value of said upper limit would increase the ratio of the crushed cotyledon that may pass through the sieve, and reduce the germ concentration. Furthermore, since the size of the germ in its width direction is usually fall into a range between about 1.5-2.0 mm, the sieve having the opening with the ratio of length/width of 2 or more, preferably 3-5 will allow recovery of non-crushed germ having its original size as well.

The shape of the opening of the sieve according to the present invention may not be limited to any particular type such as ellipse, fan, rhombus, rectangle, etc. The material and woven method of the sieve itself and its string's shape have no limitation, including, for example, a punched plate. However, a woven sieve having a rectangular opening is preferred in view of economic reasons such as a high space ratio and easiness in preparing the sieve.

By using the sieve having the rectangular opening with a particular shape described above, the soybean material having the germ concentration of 35-80 wt % may be produced with a high yield. Furthermore, in case a double-layer sieve consisting of an upper one having the rectangular opening with the particular shape according to the present invention and a lower one having a square opening with, for example, 1.4 mm or less in width, is used, small pieces of the crushed cotyledon having, for example, 1.0 mm in its diameter may pass through the lower sieve, leaving the germ fraction having a higher germ concentration of 40 wt % or more between the upper and lower sieves.

The hull of the soybean may be removed by the sorting with air before or after the sieving. Alternatively, the starting soybean material is first subjected to a sieve in order to remove a fraction of 7 mesh (2.8 mm) or more, and then applied to the above sieve having the rectanglular opening.

Oil and fat may be prepared and purified from the soybean material according to the present invention by degumming, alkali refining, bleaching and deodorizing in a conventional manner known to those skilled in the art.

Furthermore, the soybean oil and fat according to the present invention may be combined for use with various kinds of germ oils such as corn germ oil, wheat germ oil, rice germ oil, and rapeseed germ oil; and usual soybean oil, rapeseed oil and sesame oil. Furthermore, the present soybean material may be processed into an isoflavone-rich product in a conventional manner known in the art.

Foods containing the oil and fat according to the present invention include nutritional supplemented food, nutritional fortified food, and foods for specified health use. These foods may comprise any known components that are acceptable in view of food hygiene, such as food additives, foodstuffs and the like. The foods of the present invention may take various kinds of shapes or forms depending on the kinds of their components, such as mayonnaise, margarine, spread, dressing, oil for frying, bread, hamburger, solid like confectionaries, liquid, emulsion, and gel. The content of the oil and fat according to the present invention in the foods may be optionally determined by their manufacturers, depending on the kinds of the foods.

The cholesterol-lowering agent according to the present invention has an activity of lowering the level of cholesterol in the body such as in serum and liver. The present agent may comprise various kinds of pharmaceutically acceptable auxiliaries and additives, which are known to those skilled in the art, in addition to the oil and fat according to the present invention as an effective component. The auxiliaries and additives may be in the form of solid, liquid or gel depending on their kinds.

A dose of the cholesterol-lowering agent according to the present invention depends on the level of cholesterol in the body, age, sex, physical conditions of a subject, 3-30 g in terms of the soybean oil per day for an adult being appropriate. An administration route may be optionally selected such as orally, etc. Especially, the cholesterol-lowering agent according to the present invention may effectively reduce the level of cholesterol in serum.

The present invention will be further described by referring to the following example, which shall not be construed to limit the scope of the present invention at all. The term “%” in the example means “% by weight.”

### EXAMPLE 1

Selected whole soybeans were heated for 30-60 min at 80-100° C., and crudely crushed by means of a crushing roller to give a mixture of the cotyledon, germ and hull. The hull was removed by sorting with air, and a fraction of 7 mesh or more was then removed from the mixture by means of a sieve to give a smaller fraction (germ yield: 100%) comprising hull (5%), germ (12%) and cotyledon (83%).

The resulting mixture was subjected to an upper sieve having a various kinds of rectangular openings summarized in Table 1 below and a lower sieve having a square opening with 1 mm in width, followed by sorting with air of the material trapped between the upper and lower sieves to give the desired product. As comparison, a square sieve was used as the upper one as well.

TABLE 1

| Upper sieve | | Composition (%) | | | Germ | Evaluation | |
|---|---|---|---|---|---|---|---|
| Opening | Length | | Cotyl- | | Yield | Germ | Germ |
| (mm) | /Width | Germ | edon | Hull | (%) | Conc. | Yield |
| 0.3 × 4.0 | 13.3 | 25 | 74 | 1 | 10 | X | X |
| 0.5 × 4.0 | 8.00 | 42 | 57 | 1 | 82 | ◯ | ◯ |
| 0.7 × 4.0 | 5.71 | 55 | 44 | 1 | 93 | ⊚ | ◯ |
| 0.8 × 4.0 | 5.00 | 65 | 34 | 1 | 98 | ⊚ | ⊚ |
| 1.2 × 4.0 | 3.33 | 60 | 39 | 1 | 98 | ⊚ | ⊚ |
| 1.4 × 4.0 | 2.86 | 42 | 57 | 1 | 98 | ◯ | ⊚ |
| 1.7 × 4.0 | 2.35 | 30 | 69 | 1 | 98 | X | ⊚ |
| (comparative) | | | | | | | |
| 1.7 × 1.7 | 1 | 35 | 64 | 1 | 75 | X | X |
| (comparative) | | | | | | | |
| 1.4 × 1.4 | 1 | 34 | 65 | 1 | 40 | X | X |
| (comparative) | | | | | | | |

(Evaluation rate: ″⊚″ --- Excellent, ″◯″ --- Good, ″X″ --- Bad)

As seen in Table 1, use of the sieve having the opening with 0.5-1.4 mm, preferably 0.7-1.2 mm in width and with a ratio of length/width of 2 or more, preferably 3-5 made it possible to separate and concentrate a fraction of the soybean material having a high germ concentration with a high yield.

### ADVANTAGES OF THE INVENTION

Although the sieve having a rectangular opening has been widely used, its main purpose and advantage is to improve a processing capability due to its larger space ratio than that of a square opening. The other advantage of the rectangular opening is that the degree and amount of curve of strings in a length direction of the rectangular opening are less than those of the square one, making it possible to use dicker and stronger wires and to reduce clogging.

On the other hand, in the present invention, based on the findings that even the staring soybean material is crudely crushed to such a degree that the germ is completely removed from the cotyledon in a process for collecting germ, the size of the germ in its thickness direction is sufficiently smaller than that of the crushed cotyledon, it has become now possible to obtain the soybean fraction having the high germ concentration with the high yield.

Furthermore, by using the sieve having the rectangular opening particularly defined in the present invention instead of the square opening, a higher germ concentration may be realized even under crude crushing conditions in which a mingling ratio of the crushed cotyledon is small because of the soybean starting material being crushed only to its half-cut. Thus, although a small amount of the crushed cotyledon with a small diameter is produced even under the condition of the crude crushing to half-cut, it can be removed by means of the sieve having the rectangular opening according to the present invention.

What is claimed is:

1. A method for producing a soybean material having a germ concentration of 35-80 wt %, comprising crudely crushing a starting soybean material or its selected seeds from which foreign substances have been removed into a size of ½ or less of the original one, and separating and concentrating a soybean germ fraction by means of a sieve having an opening with 0.5-1.4 mm in width and with a ratio of length/width of 2 or more.

2. A method for producing a soybean material having a germ concentration of 35-80 wt %, comprising crudely crushing a starting soybean material or its selected seeds from which foreign substances have been removed into a size of from ⅛ or more to ⅖ or less of the original one, and separating and concentrating a soybean germ fraction by means of a sieve having an opening with 0.5-1.4 mm in width and with a ratio of length/width of 2 or more.

3. A method for producing a soybean material having a germ concentration of 35-80 wt %, comprising crudely crushing a starting soybean material or its selected seeds from which foreign substances have been removed into a size of ½ or less of the original one, and separating and concentrating a soybean germ fraction by means of a sieve having an opening with 0.7-1.2 mm in width and with a ratio of length/width of 2 or more.

4. A method for producing a soybean material having a germ concentration of 35-80 wt %, comprising crudely crushing a starting soybean material or its selected seeds from which foreign substances have been removed into a size of from ⅛ or more to ⅖ or less of the original one, and separating and concentrating a soybean germ fraction by means of a sieve having an opening with 0.7-1.2 mm in width and with a ratio of length/width of 2 or more.

5. A method for producing a soybean material having a germ concentration of 35-80 wt %, comprising crudely crushing a starting soybean material or its selected seeds from which foreign substances have been removed into a size of ½ or less of the original one, and separating and concentrating a soybean germ fraction by means of a sieve having an opening with 0.5-1.4 mm in width and with a ratio of length/width of 3-5.

6. A method for producing a soybean material having a germ concentration of 35-80 wt %, comprising crudely crushing a starting soybean material or its selected seeds from which foreign substances have been removed into a size of from ⅛ or more to ⅖ or less of the original one, and separating and concentrating a soybean germ fraction by means of a sieve having an opening with 0.5-1.4 mm in width and with a ratio of length/width of 3-5.

7. A method for producing a soybean material having a germ concentration of 35-80 wt %, comprising crudely crushing a starting soybean material or its selected seeds from which foreign substances have been removed into a size of ½ or less of the original one, and separating and concentrating a soybean germ fraction by means of a sieve having an opening with 0.7-1.2 mm in width and with a ratio of length/width of 3-5.

8. A method for producing a soybean material having a germ concentration of 35-80 wt %, comprising crudely crushing a starting soybean material or its selected seeds from which foreign substances have been removed into a size of from ⅛ or more to ⅖ or less of the original one, and separating and concentrating a soybean germ fraction by means of a sieve having an opening with 0.7-1.2 mm in width and with a ratio of length/width of 3-5.

9. Oil and fat obtained from the soybean material produced according to any one of claim 1-8.

10. A food containing oil and fat obtained from the soybean material produced according to any one of claim 1-8.

11. An agent for lowering cholesterol comprising as an effective component oil and fat obtained from the soybean material produced according to any one of claim 1-8.
